(19) United States
(12) Patent Application Publication
(10) Pub. No.: US 2014/0132919 A1
(43) Pub. Date: May 15, 2014

(54) OPHTHALMOLOGIC PHOTOGRAPHING APPARATUS

(71) Applicant: CANON KABUSHIKI KAISHA, TOKYO (JP)

(72) Inventor: Akihiro Kushida, Kawasaki-shi (JP)

(73) Assignee: CANON KABUSHIKI KAISHA, TOKYO (JP)

(21) Appl. No.: 14/066,970

(22) Filed: Oct. 30, 2013

(30) Foreign Application Priority Data
Nov. 9, 2012 (JP) 2012-247028

(51) Int. Cl. A61B 3/12

(52) U.S. Cl. CPC A61B 3/12
USPC 351/206

(57) ABSTRACT

Provided is an ophthalmologic apparatus including: photographing units for photographing an eye in different modes; a photographing mode switchover unit for switching the photographing units; a photographing light intensity memory unit for storing a photographing light intensity for each photographing mode; a reference light intensity setting unit for setting a reference light intensity serving as a switchover determination reference of the photographing mode; a photographing mode switchover setting unit for setting a switchover operation for each photographing mode and an operation timing at which an operation of the photographing mode is to be executed; and a control unit for controlling such that, when the photographing light intensity is equal to or higher than the reference light intensity at a predetermined operation timing, the photographing mode switchover setting unit switches the photographing mode to a photographing mode used for the photographing light intensity less than the reference light intensity.

### BACKGROUND OF THE INVENTION

1. Field of the Invention

The present invention relates to an ophthalmologic photographing apparatus.

2. Description of the Related Art

There are known non-mydriatic photographing in which fundus photographing is performed without instilling a mydriatic agent into an eye to be inspected and mydriatic photographing in which fundus photographing is performed by instilling a mydriatic agent into an eye to be inspected. In the non-mydriatic photographing, fundus photographing is performed while the eye to be inspected is naturally dilated in the dark. In order to prevent miosis of the eye to be inspected at the time of observation, infrared light is generally used for observation. Meanwhile, in the mydriatic photographing, fundus photographing is performed while the eye to be inspected is forcibly dilated using the mydriatic agent. Since miosis does not occur even when visible light is irradiated, the visible light is generally used for observation. However, in order to alleviate a burden on the eye to be inspected, infrared light may also be used for observation.

In Japanese Patent No. 4669891, in a fundus camera that can perform both non-mydriatic photographing and mydriatic photographing, in order to prevent miosis of the eye to be inspected caused by forgetting to switch from visible light observation to infrared light observation, observation light switchover control is performed.

In age-related macular degeneration, a substance called lipofuscin is accumulated in a macular region of a retinal fundus. Since the accumulated substance lipofuscin is a type of fluorescent substance, autofluorescence (or natural fluorescence) is emitted when visible light having a predetermined wavelength of 575 to 585 nm is irradiated. It is conceived that early diagnosis of the age-related macular degeneration can be made by measuring and grading a light intensity of the autofluorescence from the retinal fundus.

Japanese Patent Application Laid-open No. 2010-220670 discloses an apparatus capable of both color photographing and autofluorescence photographing in a non-mydriatic fundus camera in which the infrared light is used for observation. However, since a light intensity of autofluorescence from the fundus is very low, a high light intensity is necessary for photographing. For this reason, Japanese Patent Application Laid-Open No. 2010-259531 discloses an apparatus that captures a moving picture while exposure control is performed such that miosis of the eye to be inspected does not occur, and generates an autofluorescence photographing image by synthesizing a plurality of images.

In the non-mydriatic fundus camera capable or both color photographing and autofluorescence photographing, a higher light intensity is necessary in autofluorescence photographing rather than color photographing. When autofluorescence photographing is erroneously performed for the eye to be inspected for which color photographing is suitable, miosis occurs in the eye to be inspected, and it takes time until photographing is allowable. As a result, photographing efficiency decreases.

In Japanese Patent No. 4669891, in order to prevent miosis of the eye to be inspected, observation light switchover control is performed. However, it fails to discuss prevention of miosis of the eye to be inspected caused by erroneous photographing in a photographing mode in which a nigh light intensity is necessary.

Japanese Patent Application Laid-Open No. 2010-220670 discloses a configuration for performing both color photographing and autofluorescence photographing using a non-mydriatic fundus camera in which infrared light is used for observation. However, it fails to discuss prevention of miosis of the eye to be inspected caused by erroneous photographing in a photographing mode in which a high light intensity is necessary.

In Japanese Patent Application Laid-Open No. 2010-259531, since a moving picture is taken while exposure control is performed, and an autofluorescence image is generated by synthesizing a plurality of images, it is possible to prevent miosis of the eye to be inspected. However, an apparatus configuration becomes complicated.

### SUMMARY OF THE INVENTION

In view of the aforementioned problems, the invention provides a non-mydriatic fundus camera capable of both color photographing and autofluorescence photographing and preventing miosis of an eye to be inspected caused by erroneous autofluorescence photographing using a simple configuration.

In order to address the problems described above, an ophthalmologic photographing apparatus according to the present invention includes: a plurality of photographing units configured to photograph an eye to be inspected in different modes; a photographing mode switchover unit configured to switch the plurality of photographing units; a photographing light intensity memory unit configured to store a photographing light intensity for each photographing mode; a reference light intensity setting unit configured to set a reference light intensity serving as a switchover determination reference of the photographing mode; a photographing mode switchover setting unit configured to set a switchover operation of the photographing mode for each photographing mode and an operation timing at which an operation of the photographing mode is to be executed; and a control unit configured to perform control such that, when the photographing light intensity is equal to or higher than the reference light intensity at a predetermined operation timing of the operation timing, the photographing mode switchover setting unit switches the photographing mode to a photographing mode used for the photographing light intensity less than the reference light intensity.

An ophthalmologic photographing apparatus according to the present invention includes a plurality of photographing units configured to photograph an eye to be inspected in different modes, a photographing mode switchover unit configured to switch the plurality of photographing units, a photographing light intensity memory unit configured to store a photographing light intensity according to each photographing mode, a reference light intensity setting unit configured to set a reference light intensity serving as a switchover determination reference of the photographing mode, a photographing mode switchover setting unit configured to set a switchover operation of the photographing mode according to each of the photographing mode and an operation timing at which an operation to be performed in the photographing mode is performed, and a control unit configured to perform control such that, when the photographing light intensity is equal to or higher than the reference light intensity at a predetermined operation timing of the operation timing, the photographing mode switchover setting unit is allowed to display a message for switching to the photographing mode with the photographing light intensity less than the reference light intensity.

According to the invention, at a predetermined operation timing such as after photographing or at the time of returning to a power saving mode, a photographing mode of a photographing light intensity equal to or higher than the reference light intensity is switched to a photographing mode of a photographing light intensity less than the reference light intensity. Therefore, it is possible to prevent miosis of the eye to be inspected caused by forgetting a photographing mode switchover.

In addition, according to the invention, at a predetermined operation timing such as after photographing or at the time of returning to a power saving mode, a message for switching a photographing mode of a photographing light intensity equal to or higher than the reference light intensity to a photographing mode of a photographing light intensity less than the reference light intensity is displayed. Therefore, it is possible to prevent miosis of the eye to be inspected caused by forgetting a photographing mode switchover.

Further features of the present invention will become apparent from the following description of exemplary embodiments with reference to the attached drawings.

### BRIEF DESCRIPTION OF THE DRAWINGS

FIG. 1 is a diagram illustrating a configuration according to First and Second Embodiments.

FIG. 2 illustrates an exemplary photographing mode switchover setting according to the First Embodiment.

FIG. 3 is a flowchart according to the First Embodiment.

FIG. 4 illustrates an exemplary photographing mode switchover setting according to the Second Embodiment.

FIG. 5 is a flowchart according to Second Embodiment.

FIG. 6 illustrates an exemplary photographing mode switchover message.

FIG. 7 is a diagram illustrating a configuration according to a Third Embodiment.

FIG. 8 illustrates an exemplary mydriatic information input screen.

### DESCRIPTION OF THE EMBODIMENTS

Preferred embodiments of the present invention will now be described in detail in accordance with the accompanying drawings.

### First Embodiment

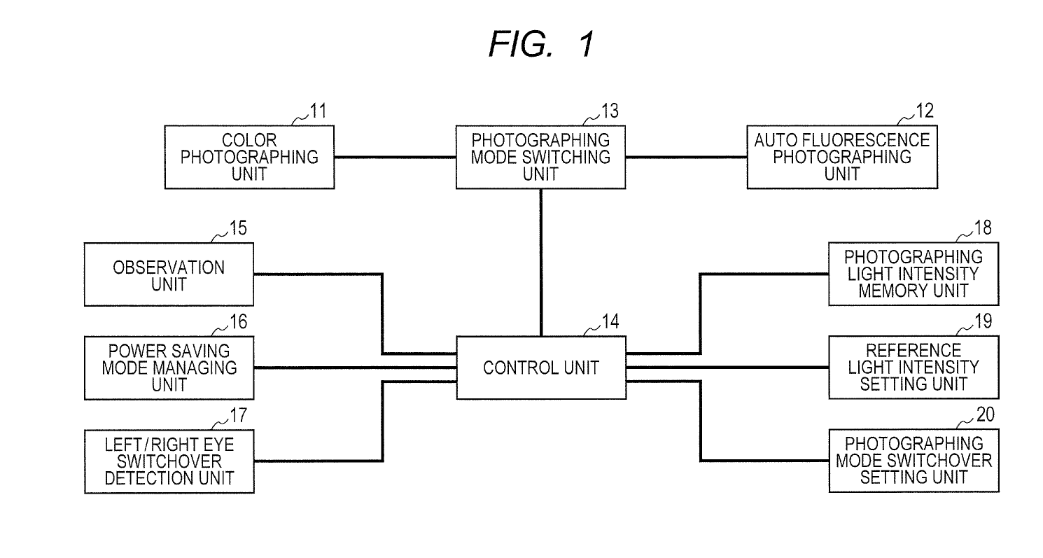

FIG. 1 is a diagram illustrating a configuration an ophthalmologic photographing apparatus according to First Embodiment.

A color photographing unit 11 configured to perform color photographing for an eye to be inspected and an autofluorescence photographing unit 12 configured to perform autofluorescence photographing for the eye to be inspected are connected to a photographing mode switchover unit 13 configured to switch between the color photographing and the autofluorescence photographing. The photographing mode switchover unit 13 is connected to a control unit 14.

An observation unit 15 configured to observe the eye to be inspected with infrared light, a power saving mode managing unit 16, and a left/right eye switchover detecting unit 17 are connected to the control unit 14. When there is no manipulation for a predetermined period or transition to a power saving mode is selected, the power saving mode managing unit 16 performs transition to the power saving mode and returns from the power saving mode through a predetermined manipulation.

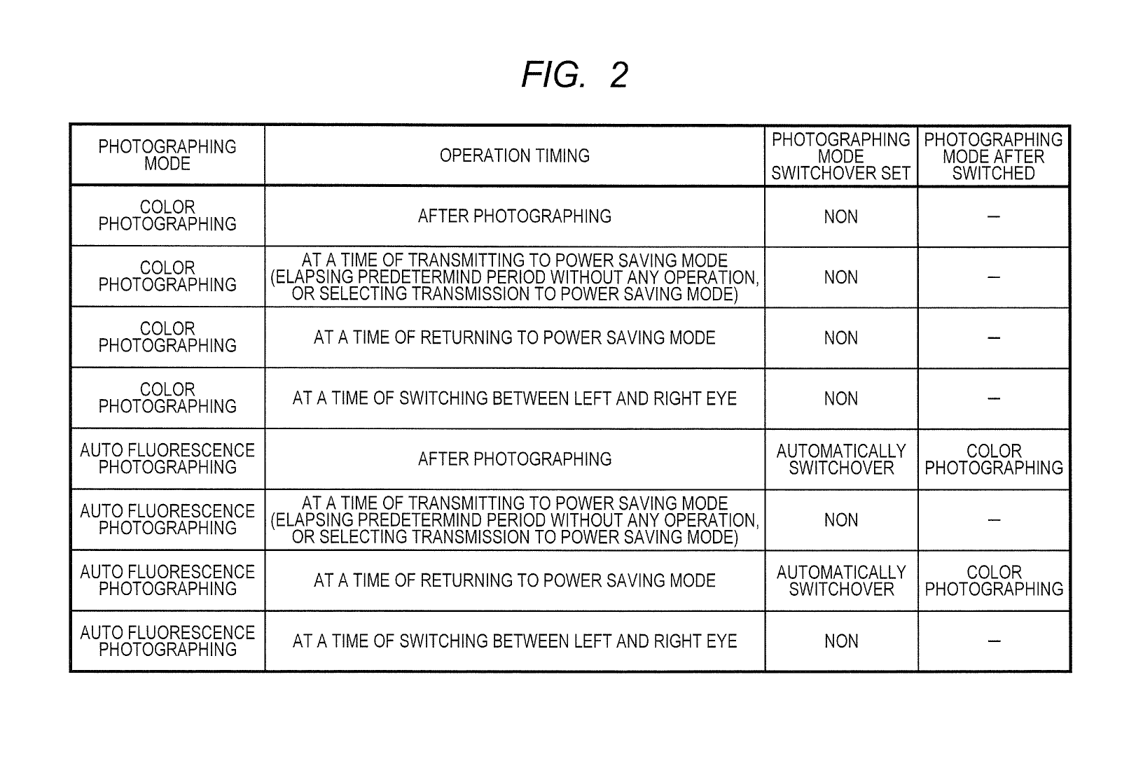

A photographing light intensity memory unit 18 configured to store photographing light intensities for each photographing mode, a reference light intensity setting unit 19 configured to set a photographing light intensity serving as a photographing mode switchover reference at predetermined operation timing, and a photographing mode switchover setting unit 20 are connected to the control unit 14. As illustrated in FIG. 2, the photographing mode switchover setting unit 20 sets, for example, whether or not the photographing mode is automatically switched, or whether or not nothing is performed, for each photographing mode and each operation timing at which various operations are performed in the photographing mode. If the photographing mode is automatically switched, the switchover target photographing mode is set.

When the timing of transition to the power saving mode is set as the operation timing, according to First Embodiment, the operation timing includes both a case where a predetermined period elapses without any manipulation and a case where the transition to the power saving mode is selected. When the timing of transition to the power saving mode or the timing of returning to the power save mode is set as the operation timing, the power saving mode managing unit 16 is necessary. In addition, the timing of switching between left eye and right eye is set as the operation timing, the left/right eye switchover detecting unit 17 is necessary. When the timing after photographing is set as the operation timing, the operation timing may include only the timing of a photographing failure. In this case, the control unit 14 determines whether or not the photographing is successful.

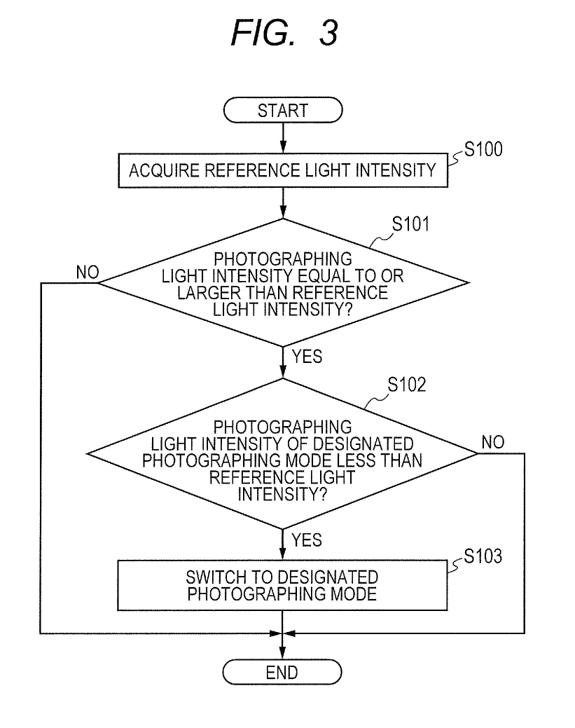

FIG. 3 illustrates a flowchart according to First Embodiment.

The process of the flowchart is executed at operation timing and in a photographing mode, in which an automatic switchover is set as the photographing mode switchover setting in the photographing mode switchover setting unit 20.

In step S100, a reference light intensity set by the reference light intensity setting unit 19 is acquired.

In step S101, it is determined whether or not a photographing light intensity of a current photographing mode stored in the photographing light intensity memory unit 18 is equal to or higher than the reference light intensity. When it is determined that the photographing light intensity of the current photographing mode is less than the reference light intensity, the process is terminated. When it is determined that the photographing light intensity of the current photographing mode is equal to or higher than the reference light intensify, the process advances to step S102. That is, the reference light intensity is used as a determination reference for switching the photographing mode.

In step S102, it is determined whether a photographing light intensity of the photographing mode designated as the switchover target photographing mode by the photographing mode switchover setting unit 20 is less than the reference light intensity. When it is determined as equal to or higher than the reference light intensity, the process is terminated. When it is determined as less than the reference light intensity, the process advances to step S103.

In step S103, the photographing mode is switched to the switchover target photographing mode designated by the photographing mode switchover setting unit 20.

Although a description has been made for color photographing and autofluorescence photographing in this embodiment, it may be similarly applied to other photographing modes such as red-free photographing.

In the photographing mode switchover setting unit 20, as a photographing mode switchover setting, the photographing light intensity of the switchover target photographing mode set to be automatically switched may be prohibited from being changed to the reference light intensity or higher. Alternatively, a warning message may be displayed when there is a try to change the photographing light intensity to the reference light intensity or higher.

As described above, in this embodiment, the color photographing and the autofluorescence photographing are employed as the photographing mode. However, the invention may be applied to a plurality of photographing modes using a plurality of photographing units that photograph the eye to be inspected in a different format. In this case, the photographing mode switchover unit 13 performs switchover between a plurality of the photographing units. In addition, the photographing light intensity is stored in the photographing light intensity memory unit 18 as a value corresponding to each photographing mode. Furthermore, as described above, the photographing mode switchover setting unit 20 sets the photographing mode switchover operation depending on each photographing mode and operation timing for executing various operations in the corresponding photographing mode.

### Second Embodiment

FIG. 1 illustrates a configuration of the ophthalmologic photographing apparatus according to Second Embodiment. Since the configuration diagram is similar to that of First Embodiment, a description will be made only for a difference from First Embodiment.

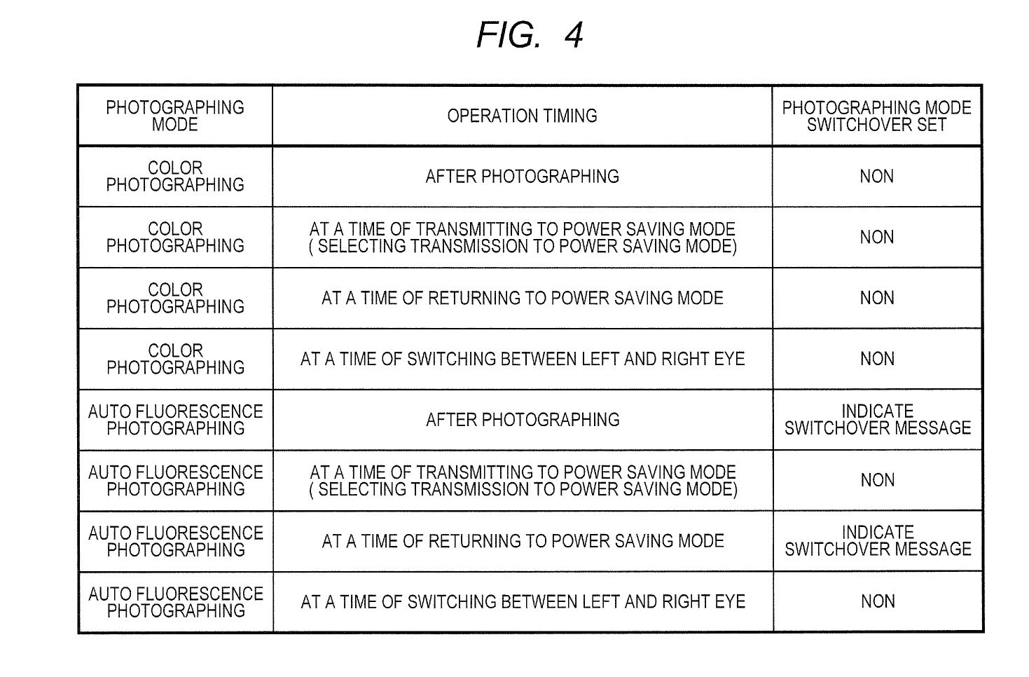

In the photographing mode switchover setting unit 20, for example, as illustrated in FIG. 4, whether or not a photographing mode switchover message is displayed or whether or not nothing is performed is set for each photographing mode and each operation timing.

When the timing of transition to the power saving mode is set as the operation timing, according to First Embodiment, the operation timing includes both a case where a predetermined period elapses without any manipulation and a case where transition to the power saving mode is selected by a predetermined operation. However, according to Second Embodiment, the operation timing includes only a case where the transition to the power saving mode is selected.

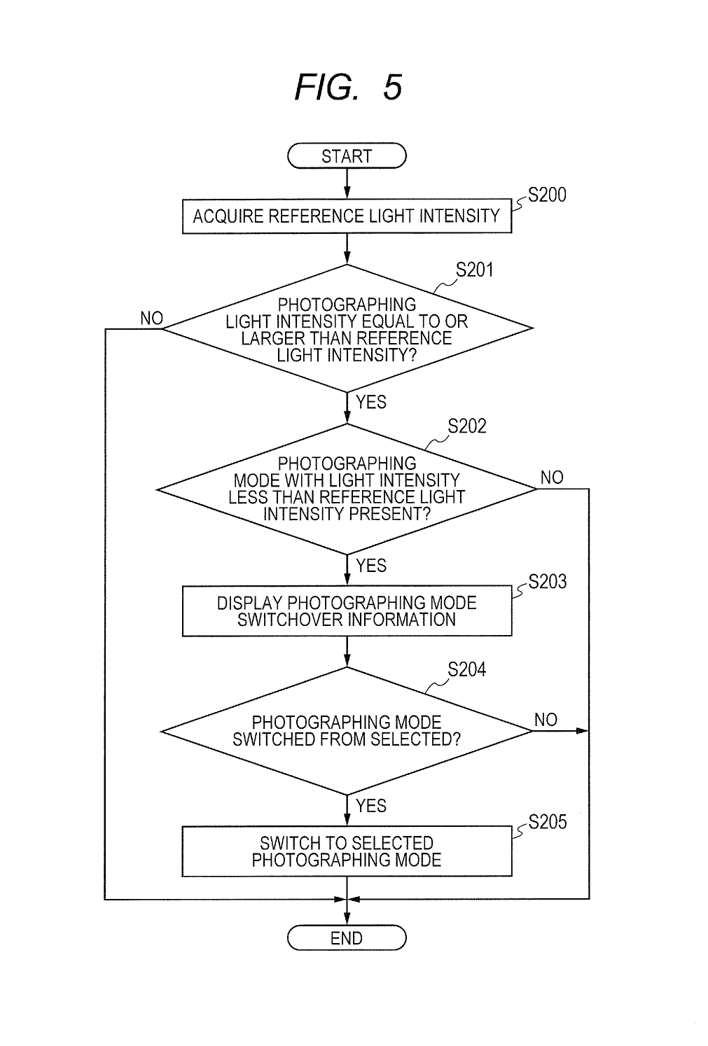

FIG. 5 illustrates a flowchart according to Second Embodiment.

The process of the flowchart is executed at operation timing in a photographing mode, in which displaying a switchover message is set as the photographing mode switchover setting in the photographing mode switchover setting unit 20.

In step S200, a reference light intensity set by the reference light intensity setting unit 19 is acquired.

In step S201, it is determined whether a photographing light intensity of the current photographing mode stored in the photographing light intensity memory unit 18 is equal to or higher than the reference light intensity. When it is determined that the photographing light intensity of the current photographing mode is less than the reference light intensity, the process is terminated. When it is determined that the photographing light intensity of the current photographing mode is equal to or higher than the reference light intensity, the process advances to step S202.

In step S202, for modes other than the current photographing mode, it is determined whether or not there is a photographing mode in which the photographing light intensity is less than the reference light intensity. When it is determined that there is no photographing mode in which the photographing light intensity is less than the reference light intensity, the process is terminated. Meanwhile, if it is determined that there is the photographing mode in which the photographing light intensity is less than the reference light intensity, the process advances to step S203.

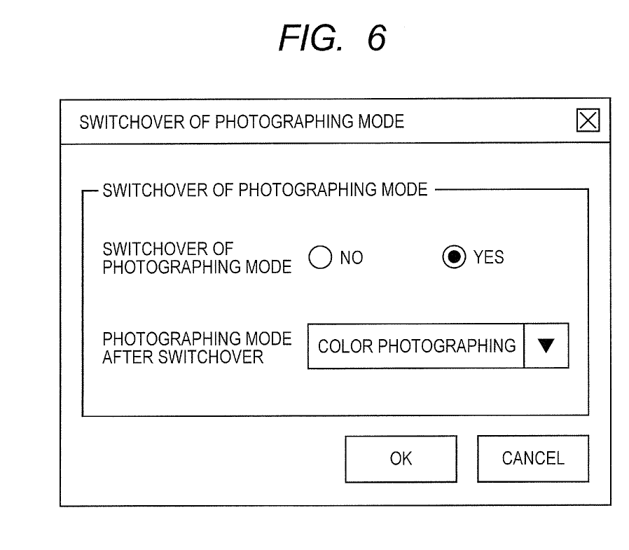

In step S203, as illustrated in FIG. 6, a photographing mode switchover message is displayed. In this message, the switchover target photographing mode may be selected from the photographing mode in which the photographing light intensity is less than the reference light intensity. When no photographing mode switchover is selected, the process is terminated. When the switchover target photographing mode is selected, the process advances to step S205.

In step S205, switching to the selected photographing mode is performed.

While the embodiment has been described for both color photographing and autofluorescence photographing, the invention may similarly apply to other photographing modes such as red-free photographing.

In the photographing mode switchover setting unit 20, when a switchover message display setting is performed at least once as the photographing mode switchover setting, a change of the photographing light intensity to the reference light intensity or higher may be prohibited in the photographing mode in which the photographing light intensity is less than the reference light intensity at that time. Alternatively, when there is a try to change the photographing light intensity to the reference light intensity or higher, a warning message may be displayed.

When there is only one photographing mode in which the photographing light intensity is less than the reference light intensity, a change of the photographing light intensity of the corresponding photographing mode to the reference light intensity or higher may be prohibited. Alternatively, when there is a try to change the photographing light intensity to the reference light intensity or higher, a warning message may be displayed.

### Third Embodiment

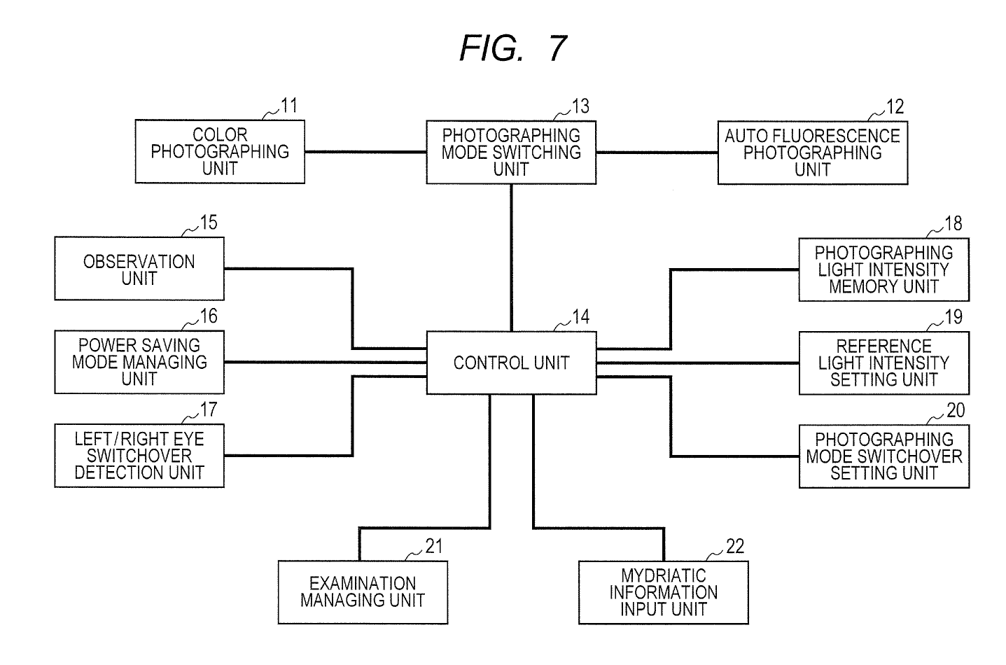

FIG. 7 is a diagram illustrating a configuration according to Third Embodiment. A description will be made only for differences from the configuration of Embodiments 1 and 2.

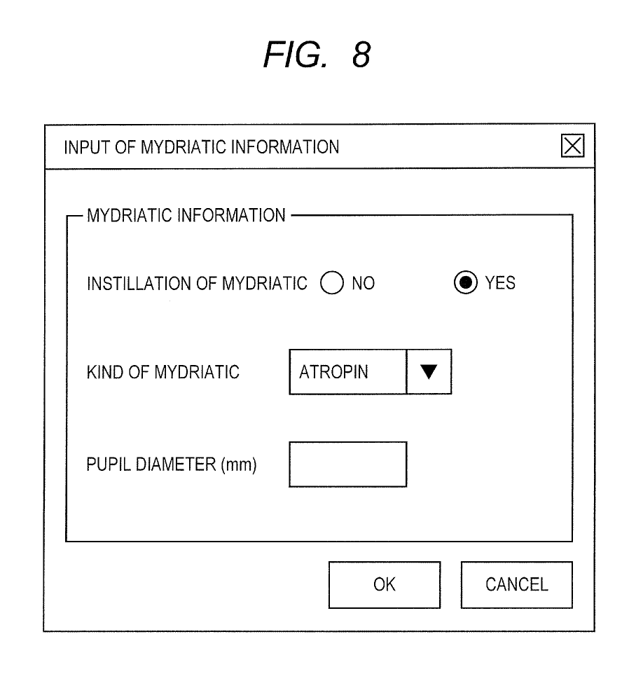

An examination managing unit 21 configured to manage the start and end of the examination of the eye to be inspected including mydriatic photographing and a mydriatic information input unit 22 configured to input whether or not a mydriatic agent is instilled into the eye to be inspected as illustrated in FIG. 8 are connected to the control unit 14.

In the photographing mode switchover setting unit 20, the end timing of the examination is set as operation timing.

According to Third Embodiment, another condition, that is, when instillation of the mydriatic agent into the eye to be inspected is not input in the mydriatic information input unit 22, is added to the process start condition of the flowcharts of FIGS. 3 and 5 described in Embodiments 1 and 2.

When no instillation of the mydriatic agent into the eye to be inspected is input, or when mydriatic information is not input, the process of the flowcharts of FIGS, 3 and 5 is executed as it is.

When instillation of the mydriatic agent into the eye to be inspected is input, the process of the flowcharts of FIGS. 3 and 5 is not performed. In this case, when the examination managing unit 21 detects examination termination, the process set for the operation timing at a time of examination termination by the photographing mode switchover setting unit 20 are performed. When an automatic switchover is set as the photographing mode switchover setting at the time of examination termination, the process of the flowchart of FIG. 3 is performed. When displaying of the switchover message is set as the photographing mode switchover setting at the time of examination termination, the process of the flowchart in FIG. 5 is performed.

### Other Embodiments

Moreover, the invention may also be implemented by performing the following process. Specifically, software (program) in which functions of the aforementioned embodiments are implemented is provided in a system or an apparatus via a network or various recording media, and a computer (for example, a CPU or an MPU) of the system or apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-247028, filed Nov. 9, 2012, which is hereby incorporated by reference herein in its entirety.

What is claimed is:

1. An ophthalmologic photographing apparatus comprising:
a plurality of photographing units configured to photograph an eye to be inspected in different modes;
a photographing mode switchover unit configured to switch the plurality of photographing units;
a photographing light intensity memory unit configured to store a photographing light intensity for each photographing mode;
a reference light intensity setting unit configured to set a reference light intensity serving as a switchover determination reference of the photographing mode;
a photographing mode switchover setting unit configured to set a switchover operation of the photographing mode for each photographing mode and an operation timing at which an operation of the photographing mode is to be executed; and
a control unit configured to perform control such that, when the photographing light intensity is equal to or higher than the reference light intensity at a predetermined operation timing of the operation timing, the photographing mode switchover setting unit switches the photographing mode to a photographing mode used for the photographing light intensity less than the reference light intensity.

2. An ophthalmologic photographing apparatus comprising:
a plurality of photographing units configured to photograph an eye to be inspected in different modes;
a photographing mode switchover unit configured to switch the plurality of photographing units;
a photographing light intensity memory unit configured to store a photographing light intensity for each photographing mode;
a reference light intensity setting unit configured to set a reference light intensity serving as a switchover determination reference of the photographing mode;
a photographing mode switchover setting unit configured to set a switchover operation of the photographing mode for each photographing mode and an operation timing at which an operation of the photographing mode is to be executed; and
a control unit configured to perform control such that, when the photographing light intensity is equal to or higher than the reference light intensity at a predetermined operation timing of the operation timing, the photographing mode switchover setting unit displays a message for switching to the photographing mode in which the photographing light intensity is less than the reference light intensity.

3. The ophthalmologic photographing apparatus according to claim 1, wherein the operation timing is a timing at which the photographing of the eye to be inspected is terminated.

4. The ophthalmologic photographing apparatus according to claim 1, further comprising a power saving mode managing unit configured to, when there is no manipulation of the photographing unit for a predetermined period, or when transition to a power saving mode is selected through a predetermined manipulation, transit the photographing mode to the power saving mode and return to the photographing mode from the power saving mode through a predetermined manipulation,
wherein the operation timing is a timing of returning to the photographing mode from the power saving mode.

5. The ophthalmologic photographing apparatus according to claim 1, further comprising a power saving mode managing unit configured to, when there is no manipulation of the photographing unit for a predetermined period, or when transition to a power saving mode is selected through a predetermined manipulation, transit the photographing mode to the power saving mode and return to the photographing mode from the power saving mode through a predetermined manipulation,
wherein the operation timing is a timing at which there is no manipulation, of the photographing unit for a predetermined period, or a timing at which transition to the power saving mode is selected through a predetermined manipulation.

6. The ophthalmologic photographing apparatus according to claim 2, further comprising a power saving mode managing unit configured to, when there is no manipulation of the photographing unit for a predetermined period, or when transition to a power saving mode is selected through a predetermined manipulation, transit the photographing mode to the power saving mode and return to the photographing mode from the power saving mode through a predetermined manipulation,
wherein the operation timing is a timing at which transition to the power saving mode is selected through a predetermined manipulation.

7. The ophthalmologic photographing apparatus according to claim 1, further comprising a left/right eye switchover detecting unit,
wherein the operation timing is a left/right eye switching timing of the eye to be inspected.

8. The ophthalmologic photographing apparatus according to claim 1, wherein, after the eye to be inspected is photographed, the control unit determines whether or not the photographing is successful, and
the operation timing is a timing at which the photographing is determined as a failure.

9. The ophthalmologic photographing apparatus according to claim 1, wherein the control unit prohibits the photographing light intensity of the photographing mode designated as a switchover target photographing mode from being changed to the reference light intensity or higher.

10. The ophthalmologic photographing apparatus according to claim 1, wherein the control unit displays a warning message when the photographing light intensity of the photographing mode designated as a switchover target photographing mode is changed to the reference light intensity or higher.

11. The ophthalmologic photographing apparatus according to claim 1, wherein the control unit does not switch the photographing mode when the photographing light intensity of the photographing mode designated as a switchover target photographing mode is equal to or higher than reference light intensity.

12. The ophthalmologic photographing apparatus according to claim 2, wherein the control unit prohibits the photographing light intensity of the photographing mode less than the reference light intensity from being changed to the reference light intensity or higher.

13. The ophthalmologic photographing apparatus according to claim 2, wherein the control unit displays a warning message when the photographing light intensity of the photographing mode less than the reference light intensity is changed to the reference light intensity or higher.

14. The ophthalmologic photographing apparatus according to claim 2, wherein the control unit does not display the photographing mode switchover message when there is no photographing mode in which a photographing light intensity is less than the reference light intensity.

15. The ophthalmologic photographing apparatus according to claim 12, wherein, when there is only one photographing mode in which a photographing light intensity is less than the reference light intensity, the control unit prohibits the photographing light intensity of the photographing mode less than the reference light intensity from being changed to the reference light intensity or higher.

16. The ophthalmologic photographing apparatus according to claim 13, wherein, if there is only one photographing mode in which a photographing light intensity is less than the reference light intensity, the control unit displays a warning message when the photographing light intensity of the photographing mode less than the reference light intensity is changed to the reference light intensity or higher.

17. The ophthalmologic photographing apparatus according to claim 1, further comprising:
an examination managing unit configured to manage a start and an end of examination including photographing of the eye to be inspected; and
a mydriatic information input unit configured to input whether or not a mydriatic agent is instilled into the eye to be inspected,
wherein, after a fact that the eye to be inspected is dilated is input, the control unit does not control switching from a photographing mode in which a photographing light intensity is equal to or higher than the reference light intensity to a photographing mode in which a photographing light intensity is less than the reference light intensity.

18. The ophthalmologic photographing apparatus according to claim 2, further comprising:
an examination managing unit configured to manage a start and an end of examination including photographing of the eye to be inspected; and
a mydriatic information input unit configured to input whether or not a mydriatic agent is instilled into the eye to be inspected;
wherein, after a fact that the eye to be inspected is dilated is input, the control unit does not display a message for switching from a photographing mode in which the photographing light intensity is equal to or higher than the reference light intensity to a photographing mode in which the photographing light intensity is less than the reference light intensity.

19. The ophthalmologic photographing apparatus according to claim 17, wherein, when a fact that the eye to be inspected is dilated is input, the operation timing is set to a timing at which the examination is terminated.

20. The ophthalmologic photographing apparatus according to claim 18, wherein, when a fact that the eye to be inspected is dilated is input, the operation timing is set to a timing at which the examination is terminated.
